SEQUENCE LISTING (32 sequences)

SEQ ID NO: 1 - protein, 153 aa, Homo Sapiens
  hu-IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 153 aa, Homo Sapiens [sequence omitted]

SEQ ID NO: 3 - protein, 177 aa, Homo Sapiens
  hu-IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - protein, 144 aa, Homo Sapiens
  hu-IL-9
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 5 - protein, 162 aa, Homo Sapiens
  hu-IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 162 aa, Homo Sapiens
  hu-IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 7 - protein, 177 aa, Homo Sapiens
  hu-IL-25
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 8 - protein, 270 aa, Homo Sapiens
  hu-IL-33
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 9 - DNA, 462 bp, Homo Sapiens
  hu-IL-2
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 10 - DNA, 462 bp, Homo Sapiens
  hu-IL-4
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 11 - DNA, 534 bp, Homo Sapiens
  hu-IL-7
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 12 - DNA, 435 bp, Homo Sapiens
  hu-IL-9
atgcttctggccatggtccttacctctgccctgctcctgtgctccgtggcaggccaggggtgtccaaccttggcggggatcctggacatcaacttcctcatcaacaagatgcaggaagatccagcttccaagtgccactgcagtgctaatgtgaccagttgtctctgtttgggcattccctctgacaactgcaccagaccatgcttcagtgagagactgtctcagatgaccaataccaccatgcaaacaagatacccactgattttcagtcgggtgaaaaaatcagttgaagtactaaagaacaacaagtgtccatatttttcctgtgaacagccatgcaaccaaaccacggcaggcaacgcgctgacatttctgaagagtcttctggaaattttccagaaagaaaagatgagagggatgagaggcaagatatga

SEQ ID NO: 13 - DNA, 489 bp, Homo Sapiens
  hu-IL-15
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 14 - DNA, 489 bp, Homo Sapiens
  hu-IL-21
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 15 - DNA, 534 bp, Homo Sapiens
  hu-IL-25
atgagggagcgacccagattaggtgaggacagttctctcattagccttttcctacaggtggttgcattcttggcaatggtcatgggaacccacacctacagccactggcccagctgctgccccagcaaagggcaggacacctctgaggagctgctgaggtggagcactgtgcctgtgcctcccctagagcctgctaggcccaaccgccacccagagtcctgtagggccagtgaagatggacccctcaacagcagggccatctccccctggagatatgagttggacagagacttgaaccggctcccccaggacctgtaccacgcccgttgcctgtgcccgcactgcgtcagcctacagacaggctcccacatggacccccggggcaactcggagctgctctaccacaaccagactgtcttctaccggcggccatgccatggcgagaagggcacccacaagggctactgcctggagcgcaggctgtaccgtgtttccttagcttgtgtgtgtgtgcggccccgtgtgatgggctag

SEQ ID NO: 16 - DNA, 813 bp, Homo Sapiens
  hu-IL-33
atgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttag

SEQ ID NO: 17 - protein, 752 aa, Artificial sequence
  HSA-IL-7 fusion protein
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 745 aa, Artificial sequence
  IL-25-HSA fusion protein
YSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 19 - protein, 396 aa, Artificial sequence
  IgG4mFc-IL-7 fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 389 aa, Artificial sequence
  IgG4mFc-IL-25 fusion protein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 21 - protein, 8 aa, Artificial sequence
  uPA sensitive cleavage sequence
LSGRSDNH

SEQ ID NO: 22 - protein, 4 aa, Artificial sequence
  cysteine protease sensitive cleavage sequence
AANL

SEQ ID NO: 23 - protein, 4 aa, Artificial sequence
  cysteine protease sensitive cleavage sequence
AANV

SEQ ID NO: 24 - protein, 6 aa, Artificial sequence
  MMP-1 sensitive cleavage sequence
PLGLWA

SEQ ID NO: 25 - protein, 7 aa, Artificial sequence
  MMP-2/9 sensitive cleavage sequence
PAALVGA

SEQ ID NO: 26 - protein, 10 aa, Artificial sequence
  MMP-14 sensitive cleavage sequence
SGRIGFLRTA

SEQ ID NO: 27 - protein, 4 aa, Artificial sequence
  Cathepsin B sensitive cleavage sequence
GFLG

SEQ ID NO: 28 - protein, 5 aa, Artificial sequence
  flexible linker
GGGGS

SEQ ID NO: 29 - protein, 5 aa, Artificial sequence
  alpha-helix linker
EAAAK

SEQ ID NO: 30 - protein, 338 aa, Artificial sequence
  IL-25-IL7 recombinant protein
YSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMGGGSGLSGRSDNHGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSCRGDKGPDC

SEQ ID NO: 31 - protein, 489 aa, Artificial sequence
  IL-25-IL-7-IL-2 recombinant protein
YSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMGGGSGLSGRSDNHGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGSGLSGRSDNHGGGGSAPTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSCRGDKGPDC

SEQ ID NO: 32 - protein, 6 aa, Artificial sequence
  MMP-14 sensitive cleavage sequence
PAGLVG

(19) United States
(12) Patent Application Publication
(10) Pub. No.: US 2019/0216898 A1
(43) Pub. Date: Jul. 18, 2019

(54) Interleukin Combination and Use Thereof

(71) Applicants: Mulin WANG, Shenmu Town, Shenmu County, Shaanxi (CN); Huanhuan FENG, Haikou City, Hainan Province (CN)

(72) Inventors: Mulin WANG, Shenmu Town (CN); Huanhuan FENG, Haikou City (CN); Zhenying ZHNAG, Xiangcheng City (CN); Linchong WANG, Beijing City (CN); Xudong ZHU, Tianjin City (CN)

(21) Appl. No.: 16/099,280

(22) PCT Filed: May 5, 2017
(86) PCT No.: PCT/CN2017/083203

§ 371 Date: Nov. 6, 2018

(30) Foreign Application Priority Data
May 6, 2016 (CN) 201610297836.1

(51) Int. Cl. A61K 38/20; A61P 35/00; C12N 15/62; C12N 15/86; A61K 47/65; A61K 35/76

(52) U.S. Cl. CPC A61K 38/2086; A61P 35/00; C12N 15/62; C12N 15/86; A61K 35/76; A61K 38/2026; A61K 38/2046; A61K 38/206; A61K 47/65; A61K 38/2013

(57) ABSTRACT

Disclosed are an interleukin combination or fusion proteins for preventing and/or treating malignant tumors, and various products prepared with same and the use thereof.

### FIELD

The present application relates to interleukin combinations or recombinant proteins for the prevention and/or treatment of malignant tumors, various products formed therefrom, and use thereof.

### BACKGROUND

γc-cytokine, also known as γc-family cytokine or common cytokine receptor gamma-chain family, consists of six members, namely IL-2, IL-4, IL-7, IL-9, IL-15 and IL-21. All members of this family conduct signal through a receptor complex containing a common cytokine receptor gamma chain subunit. The receptors for IL-4, IL-7, IL-9, and IL-21 are heterodimeric complexes composed of a common gamma chain and a unique cytokine-specific subunit; the receptors for IL-2 and IL-15 are heterotrimeric complexes composed of IL-2Rα/IL-15Rα, IL-2/IL-15 Rβ, and a common γ-chain.

Signaling pathways for activation of the common cytokine receptor γ-chain family include the PI3-K-AKT signaling pathway, the Ras-MAPK signaling pathway, and the JAK-STAT signaling pathway. Cytokines of this family are critical for the establishment and maintenance of normal immune system functions and have unique and overlapping effects on many types of cells including T cells, B cells, natural killer cells, mast cells, and myeloid and erythroid progenitor cells.

IL-2 was originally identified as a T cell growth factor and has been shown to play a crucial role in maintaining T cell homeostasis and preventing autoreactivity. IL-4 is necessary for Th2 and Th9 cell differentiation, regulation of immunoglobulin class switching, and promotion of mast cell survival and proliferation. IL-7 is necessary for T cell development and homeostasis proliferation, mouse B cell development, and memory T cell production. IL-9 promotes the growth of mouse T cells and mast cells, regulates the production of immunoglobulin by B cells, enhances the expression of proteases by mast cells, promotes goblet cell proliferation and mucus secretion. IL-15 is necessary for NK cell development, survival and activation, NKT cell and intraepithelial lymphocyte homeostasis, and maintenance of primary and memory CD8⁺ T cells. The recently reported family member IL-21 is necessary for Th17 cell differentiation and follicular helper T cell production. In addition, it also regulates B cell activity and cytotoxicity of CD8⁺ T cells and NK cells.

IL-25, a pro-inflammatory cytokine, is a member of the IL-17 family, which is highly expressed in certain organs such as testis, prostate, and spleen, and is lowly expressed in other organs including normal mammary glands. Although IL-25 is structurally related to the IL-17 family, it is distinct from IL-17 and other family members in biological functions. IL-25 is highly expressed in activated Th2 cells and non-T cells, has a Th2 cytokine-promoting response, has a protective immune response against helminth infection, and negatively regulates Th17 function in autoimmune inflammation.

IL-33 is a new member of the IL-1 family discovered in 2005. It activates mast cells, lymphocytes and eosinophils to produce class Th2 cytokines, and plays an important role in inflammation, infection, and autoimmune diseases.

IL-25 and IL-33 are important for the body's anti-helminth immunity, anti-fungal infections, allergic inflammation and mucosal function. Certain stimuli, such as fungal infections, can trigger the release of IL25 and IL33 from epithelial cells, both of which activate type 2 Innate lymphoid cells (ILC2) and cause a population of ILC2 to expand and to release IL5 and IL13, which in turn promote amplification and activation of eosinophils, although ILC2s activated by IL-25 and IL-33 are different. Activated eosinophils can produce cytotoxic effects on target cells. The ILC2 cell population itself has strong plasticity. For example, IL13⁻ILC2 in peripheral blood can obtain the ability to produce IFNγ, which may be related to autoimmune disease reaction such as colitis.

### SUMMARY

In a first aspect, the present disclosure provides a pharmaceutical combination for preventing and/or treating a malignant tumor comprising:

i) at least one of γc-cytokine or an active part or variant thereof, or a vector or a cell or a population of cells capable of producing at least one of γc-cytokine or an active part or variant thereof, or a nucleic acid molecule capable of encoding at least one of γc-cytokine or an active part or variant thereof; and

ii) at least one of IL-25 or IL-33 or an active part or variant thereof, or a vector or a cell or a population of cells capable of producing at least one of IL-25 or IL-33 or an active part or variant thereof, or a nucleic acid molecule capable of encoding at least one of IL-25 or IL-33 or an active part or variant thereof.

In one embodiment, at least one of γc-cytokine in the pharmaceutical combination provided in the present disclosure comprises IL-2, IL-4, IL-7, IL-9, IL-15 or IL-21. In another embodiment, at least one of γc-cytokine in the pharmaceutical combination provided in the present disclosure comprises IL-2, IL-7, IL-9 or IL-15. In still another embodiment, at least one of γc-cytokine in the pharmaceutical combination provided in the present disclosure comprises IL-7 or IL-2.

Thus, in certain embodiments, the pharmaceutical combination provided in the present disclosure includes or consists of:

i) at least one of IL-2, IL-4, IL-7, IL-9, IL-15 or IL-21 or an active part or variant thereof, or a vector or a cell or a population of cells capable of producing at least one of IL-2, IL-4, IL-7, IL-9, IL-15 or IL-21 or an active part or variant thereof, or a nucleic acid molecule capable of encoding at least one of IL-2, IL-4, IL-7, IL- 9, IL-15 or IL-21 or an active part or variant thereof; and

ii) at least one of IL-25 or IL-33 or an active part or variant thereof, or a vector or a cell or a population of cells capable of producing at least one of IL-25 or IL-33 or an active part or variant thereof, or a nucleic acid molecule capable of encoding at least one of IL-25 or IL-33 or an active part or variant thereof.

In a second aspect, the present disclosure provids a kit or a pharmaceutical composition for preventing and/or treating a malignant tumor comprising:

a first component: at least one of γc-cytokine or an active part or variant thereof, or a vector or a cell or a population of cells capable of producing at least one of γc-cytokine or an active part or variant thereof, or a nucleic acid molecule capable of encoding at least one of γc-cytokine or an active part or variant thereof; and

a second component: at least one of IL-25 or IL-33 or an active part or variant thereof, or a vector or a cell or a population of cells capable of producing at least one of IL-25 or IL-33 or an active part or variant thereof, or a nucleic acid molecule capable of encoding at least one of IL-25 or IL-33 or an active part or variant thereof.

In a particular embodiment, the first component and the second component in the kit provided in the present disclosure are present in separate pharmaceutical compositions; or present in the same pharmaceutical composition.

In a third aspect, the present disclosure provides a recombinant protein for preventing and/or treating a malignant tumor comprising:

at least one of γc-cytokine or an active part or variant thereof; and

at least one of IL-25 or IL-33 or an active part or variant thereof.

In one embodiment, at least one of γc-cytokine in the recombinant protein provided in the present disclosure comprises IL-2, IL-4, IL-7, IL-9, IL-15 or IL-21. In another embodiment, at least one of γc-cytokine in the recombinant protein provided in the present disclosure comprises IL-2, IL-7, IL-9 or IL-15. In still another embodiment, at least one of γc-cytokine in the recombinant protein provided in the present disclosure comprises IL-7 or IL-2.

In one embodiment, the recombinant protein provided in the present disclosure comprises:

an amino acid sequence as set forth in any one of SEQ ID NOs: 1-6 or an active part or variant thereof, or an amino acid sequence having at least 70%, 80%, 90%, 95% or 99% identity with an amino acid sequence as set forth in any one of SEQ ID NOs: 1-6 or an active part or variant thereof; and

an amino acid sequence as set forth in SEQ ID NO: 7 or 8 or an active part or variant thereof, or an amino acid sequence having at least 70%, 80%, 90%, 95% or 99% identity with the amino acid sequence as set forth in SEQ ID NO: 7 or 8 or an active part or variant thereof.

In one embodiment, the recombinant protein provided in the present disclosure comprises one or more linkers, preferably peptide linkers that are cleavable in vivo or intratumorally.

In one embodiment, the linker is selected from the group consisting of one or more of: uPA linker (urokinase-type plasminogen activator substrate sequence, SEQ ID NO: 21), legumain linker (cysteine protease, SEQ ID NOs: 22 and 23), MMP-1 linker (SEQ ID NO: 24), MMP-2/9 linker (SEQ ID NO: 25), MMP-14 linker (SEQ ID NO: 26 and 32), substrate peptide linker of cathepsin B (SEQ ID NO: 27), glycine-serine linker (GGGGS)ₙ (n=1-3) (SEQ ID NO: 28), α-helix forming peptide linker (EAAAK)ₙ (n=2-5) (SEQ ID NO: 29), in vivo cleavable disulfide bond linker.

In one embodiment, the recombinant protein provided in the present disclosure comprises the sequence as set forth in SEQ ID NO: 30 or 31.

In a fourth aspect, the present disclosure provides a recombinant nucleic acid molecule for preventing and/or treating a malignant tumor comprising:

a first nucleotide sequence selected from the group consisting of: a nucleotide sequence as set forth in any one of SEQ ID NOs: 9-14 or a variant thereof, or a nucleotide sequence having at least 70%, 80%, 90%, 95% or 99% identity with the nucleotide sequence as set forth in any one of SEQ ID NOs: 9-14 or variant thereof ; and/or

a second nucleotide sequence selected from the group consisting of: a nucleotide sequence as set forth in SEQ ID NOs: 15 or 16 or a variant thereof, or a nucleotide sequence having at least 70%, 80%, 90%, 95% or 99% identity with the nucleotide sequence as set forth in SEQ ID NOs: 15 or 16 or a variant thereof.

In a fifth aspect, the present disclosure provides a vector for preventing and/or treating a malignant tumor, comprising:

a first nucleotide sequence selected from the group consisting of: a nucleotide sequence as set forth in any one of SEQ ID NOs: 9-14 or a variant thereof, or a nucleotide sequence having at least 70%, 80%, 90%, 95% or 99% identity with the nucleotide sequence as set forth in any one of SEQ ID NOs: 9-14 or variant thereof; and/or

a second nucleotide sequence selected from the group consisting of: a nucleotide sequence as set forth in SEQ ID NO: 15 or 16 or a variant thereof, or a nucleotide sequence having at least 70%, 80%, 90%, 95% or 99% identity with the nucleotide sequence as set forth in SEQ ID NO: 15 or 16 or a variant thereof.

In one embodiment, the vector provided in the present disclosure is selected from a plasmid or viral vector, including but not limited to: an adenoviral vector, an adeno-associated viral vector, a retroviral vector, a lentiviral vector, a sleeping beauty or a PiggyBac transposon system, a CRISPR/Cas9 knock-in system or any other suitable vectors.

In another embodiment, the first nucleotide sequence and the second nucleotide sequence provided in the present disclosure are present in separate vectors; or wherein the first nucleotide sequence and the second nucleotide sequence are present in the same vector.

In a sixth aspect, the present disclosure provides a cell or a population of cells for preventing and/or treating a malignant tumor, which is capable of producing the recombinant protein provided in the present disclosure, or comprises the recombinant nucleic acid molecule or vector described in the present disclosure.

In one embodiment, the cell or population of cells provided in the present disclosure is selected from the group consisting of an autologous cell, an allogeneic cell, including but not limited to dendritic cells, T cells, macrophages, eosinophils; transformed homologous or xenogeneic cells, including but not limited to Chinese hamster ovary cells (CHO), baby hamster kidney cells (BHK), human embryonic kidney HEK293 derived cells or any other suitable cells.

In another embodiment, the cell or population of cells provided in the present disclosure can be microencapsulated, for example, the transformed homologous or xenogeneic cells provided in the present disclosure can be microencapsulated. Alternatively, the cell or population of cells provided in the present disclosure can be microencapsulated with a hydrogel such as sodium alginate.

In still another embodiment, the recombinant protein provided in the present disclosure is produced by expression of different cells or populations of cells, or by expression of the same cell or population of cells; or the recombinant nucleic acid molecule or vector provided in the present disclosure is comprised in different cells or populations of cells, or comprised in the same cell or population of cells.

In a seventh aspect, the present disclosure provides a method for preventing and/or treating a malignant tumor comprising administering to individuals in need thereof a prophylactically and/or therapeutically effective amount of the pharmaceutical combination provided in the present disclosure, or the kit provided in the present disclosure, or the recombinant protein provided in the present disclosure, or the recombinant nucleic acid molecule provided in the present disclosure, or the vector provided in the present disclosure, or the cell or population of cells provided in the present disclosure.

In one embodiment, when the recombinant protein provided in the present disclosure is administered to individuals in need thereof, the dose administered is 0.0001 to 10 mg/kg body weight per time, once every 0-30 days. In another embodiment, the required corresponding dose infused by an automated timed quantitative subcutaneous continuous infusion pump, such as an insulin pump, can be selected.

In another embodiment, when the vector provided in the present disclosure, such as a virus, is intratumorally administered to individuals in need thereof, the dose administered is 10⁸-10¹³ pfu virus/tumor/time; once every 0-30 days.

In yet another embodiment, when the cell or population of cells provided in the present disclosure is administered to individuals in need thereof, the dose administered is 10⁵-10¹¹ cells/kg body weight/time, once every 0-100 days.

In an eighth aspect, the present disclosure provides the use of the following combination in the prevention and/or treatment of a malignant tumor, or in the manufacture of a medicament for the prevention and/or treatment of a malignant tumor:

i) at least one of γc-cytokine or an active part or variant thereof, or a vector or a cell or a population of cells capable of producing at least one of γc-cytokine or an active part or variant thereof, or a nucleic acid molecule capable of encoding at least one of γc-cytokine or an active part or variant thereof; and

ii) at least one of IL-25 or IL-33 or an active part or variant thereof, or a vector or a cell or a population of cells capable of producing at least one of IL-25 or IL-33 or an active part or variant thereof, or a nucleic acid molecule capable of encoding at least one of IL-25 or IL-33 or an active part or variant thereof.

In the above aspects, at least one of γc-cytokine provided in the present disclosure comprises IL-2, IL-4, IL-7, IL-9, IL-15 or IL-21; preferably IL-2, IL-7, IL-9 or IL-15; more preferably IL-7 or IL-2.

In the above aspects, at least one of γc-cytokine provided in the present disclosure, i.e., IL-2, IL-4, IL-7, IL-9, IL-15 or IL-21, or an active part or variant thereof, and/or the at least one of IL-25 or IL-33 or an active part or variant thereof, and/or the recombinant protein provided in the present disclosure further comprises one or more modifications selected from the group consisting of: fusion with human immunoglobulin Fc fragment or a variant thereof; fusion with human serum albumin or a variant thereof; fusion with a tumor-penetrating peptide; fusion with a tumor-targeting single-chain or single-domain antibody; PEGylation; HESylation; Xtenylation; PASylation; or any other suitable chemical modifications.

In the above aspects, the involved malignant tumor is selected from the group consisting of: fibrosarcoma, mucinous sarcoma, liposarcoma, chondrosarcoma, osteosarcoma, chordoma, angiosarcoma, endothelial sarcoma, lymphangiosarcoma, lymphangial endothelial sarcoma, synovial sarcoma, mesothelioma, Ewing's tumor, leiomyosarcoma, rhabdomyosarcoma, colon cancer, pancreatic cancer, breast cancer, ovarian cancer, prostate cancer, squamous cell carcinoma, basal cell carcinoma, adenocarcinoma, sweat gland cancer, sebaceous gland cancer, papillary cancer, papillary adenocarcinoma, carcinoma, bronchial carcinoma, medullary carcinoma, renal cell carcinoma, liver cancer, cholangiocarcinoma, choriocarcinoma, seminoma, embryonal carcinoma, nephroblastoma, cervical cancer, testicular tumor, lung cancer, small cell lung cancer, bladder cancer, epithelial cancer, glioma, astrocytoma, medulloblastoma, craniopharyngioma, ependymoma, pineal tumor, hemangioblastoma, acoustic neuroma, meningioma, oligodendroglioma, melanoma, neuroblastoma, and retinoblastoma.

### BRIEF DESCRIPTION OF THE DRAWINGS

FIG. 1 shows a flow chart of drug screening for B16F10 melanoma cytokine combination therapy. A is a retroviral vector constructed with mouse cytokines; B shows that HEK293T cells co-transfected with retroviral vector and packaging helper plasmid pCL-Ampho produce retroviral particles capable of expressing cytokines; C shows generation and enrichment of cell pools for cytokine production. B16F10 cells transduced with corresponding retroviruses were enriched by screening with puromycin; D relates to verifying the effects of cytokines and combinations thereof on tumor growth. The primary B16F10 cells were injected into the left side of mice as response tumors, and a cell pool for producing single cytokine or two cell pools for producing different cytokines were injected into the right side as cytokine tumors.

FIG. 2 exemplarily shows the results of the inhibitory effects of some cytokines on tumor growth (n=4 per group). Primary B16F10 cells or B16F10 transduced with empty retroviral vector are used in the control group. The tumor was removed and weighed 16 days after inoculating cells.

FIG. 3 shows that tumor growth dynamics, suggesting a synergistic anti-tumor effect of the combination of IL-7 and IL-25 in C57BL/6 mice. 5×10⁵ B16F10 primary cells were injected intradermally (i.d.) into the left side of C57BL/6 mice (as response tumors), whereas a cell pool for producing IL-7 or IL-25 or a mixed cell pool for producing the two cytokines (1 million each) was injected intradermally into the right side (as a drug tumor). The area of the response tumor (A) and the area of the cytokine-producing tumor (B) (n=10 per group) were measured 4 days after tumor cell injection.

FIG. 4 shows the Coomassie brilliant blue staining map of the recombinant protein with 6× His tag expressed in E. coli BL21 (DE3) (10 μg protein per lane), where A is IL-7, B is IL-25, and C is the therapeutic response dynamics of tumors to hu-IL-7, hu-IL-25 or the combination of huIL-7+huIL-25. 5×10⁵ B16F10 primary cells were injected intradermally on both sides of C57BL/6 mice (10 in each group). After 24 hours, the mice were injected intraperitoneally twice daily with IL-7, IL-25, IL-7+ IL-25 dissolved in PBS (5 μg each/time). The tumor area was measured daily 5 days after tumor cell injection.

### DESCRIPTION OF SEQUENCES

Unless otherwise indicated, the amino acid sequences of the signal peptides or corresponding nucleotide sequences thereof are shown in bold in the following sequences.

SEQ ID NO: 1 shows the amino acid sequence of
human IL-2.
MYRMQLLSCIALSLALVTNSAPTSSSTKKTQLQLEHLLLDLQMILNG
INNYKNPKLTRMLTFKFYMPKKATELKHLQCLEEELKPLEEVLNLAQ
SKNFHLRPRDLISNINVIVLELKGSETTFMCEYADETATIVEFLNRW
ITFCQSIISTLT
SEQ ID NO: 2 shows the amino acid sequence of
human IL-4.
MGLTSQLLPPLFFLLACAGNFVHGHKCDITLQEIIKTLNSLTEQKTL
CTELTVTDIFAASKNTTEKETFCRAATVLRQFYSHHEKDTRCLGATA
QQFHRHKQLIRFLKRLDRNLWGLAGLNSCPVKEANQSTLENFLERLK
TIMREKYSKCSS
SEQ ID NO: 3 shows the amino acid sequence of
human IL-7, in which the underlined amino acid
residues in italics (positions 96-114) can be
deleted from hu-IL-7 sequence and the resultant
sequence is a splice variant.
MFHVSFRYIF GLPPLILVLL PVASSDCDIE GKDGKQYESV
LMVSIDQLLD SMKEIGSNCL NNEFNFFKRH ICDANKEGMF
LFRAARKLRQ FLKMNSTGDF DLHLLKVSEG TTILLNCTGQ
VKGRKPAALG EAQPTKSLEE NKSLKEQKKL NDLCFLKRLL
QEIKTCWNKI LMGTKEH
SEQ ID NO: 4 shows the amino acid sequence of
human IL-9.
MLLAMVLTSALLLCSVAGQGCPTLAGILDINFLINKMQEDPASKCHC
SANVTSCLCLGIPSDNCTRPCFSERLSQMTNTTMQTRYPLIFSRVKK
SVEVLKNNKCPYFSCEQPCNQTTAGNALTFLKSLLEIFQKEKMRGMR
GKI
SEQ ID NO: 5 shows the amino acid sequence of
human IL-15.
MRISKPHLRSISIQCYLCLLLNSHFLTEAGIHVFILGCFSAGLPKTE
ANWVNVISDLKKIEDLIQSMHIDATLYTESDVHPSCKVTAMKCFLLE
LQVISLESGDASIHDTVENLIILANNSLSSNGNVTESGCKECEELEE
KNIKEFLQSFVHIVQMFINTS
SEQ ID NO: 6 shows the amino acid sequence of
human IL-21.
MRSSPGNMERIVICLMVIFLGTLVHKSSSQGQDRHMIRMRQLIDIVD
QLKNYVNDLVPEFLPAPEDVETNCEWSAFSCFQKAQLKSANTGNNER
IINVSIKKLKRKPPSTNAGRRQKHRLTCPSCDSYEKKPPKEFLERFK
SLLQKMIHQHLSSRTHGSEDS
SEQ ID NO: 7 shows the amino acid sequence of
human IL-25, in which the signal peptide
sequence in bold italics in a splice variant
hu-IL-25 is different and can be replaced with
the amino acid Y, but the mature IL-25 sequence
is identical.
M [symbol] [symbol] [symbol] QVVAFLAMVMGTHTYSHWPSCCPSKGQD
TSEELLRWSTVPVPPLEPARPNRHPESCRASEDGPLNSRAISPWRYE
LDRDLNRLPQDLYHARCLCPHCVSLQTGSHMDPRGNSELLYHNQTVE
YRRPCHGEKGTHKGYCLERRLYRVSLACVCVRPRVMG
SEQ ID NO: 8 shows the amino acid sequence of
human IL-33, in which shown in bold is the
amino acid sequence comprised in the propeptide.
MKPKMKYSTNKISTAKWKNTASKALCFKLGKSQQKAKEVCPMYFMKL
RSGLMIKKEACYFRRETTKRPSLKTGRKHKRHLVLAACQQQSTVECF
AFGISGVQKYTRALHDSSITGISPITEYLASLSTYNDQSITFALEDE
SYEIYVEDLKKDEKKDKVLLSYYESQHPSNESGDGVDGKMLMVTLSP
TKDFWLHANNKEHSVELHKCEKPLPDQAFFVLHNMHSNCVSFECKTD
PGVFIGVKDNHLALIKVDSSENLCTENILFKLSET
SEQ ID NO: 9 shows the nucleotide sequence of
human IL-2.
atgtacaggatgcaactcctgtcttgcattgcactaagtcttgcact
tgtcacaaacagtgcacctacttcaagttctacaaagaaaacacagc
tacaactggagcatttactgctggatttacagatgattttgaatgga
attaataattacaagaatcccaaactcaccaggatgctcacatttaa
gttttacatgcccaagaaggccacagaactgaaacatcttcagtgtc
tagaagaagaactcaaacctctggaggaagtgctaaatttagctcaa
agcaaaaactttcacttaagacccagggacttaatcagcaatatcaa
cgtaatagttctggaactaaagggatctgaaacaacattcatgtgtg
aatatgctgatgagacagcaaccattgtagaatttctgaacagatgg
attaccttttgtcaaagcatcatctcaacactgacttga
SEQ ID NO: 10 shows the nucleotide sequence of
human IL-4.
atgggtctcacctcccaactgcttccccctctgttcttcctgctagc
atgtgccggcaactttgtccacggacacaagtgcgatatcaccttac
aggagatcatcaaaactttgaacagcctcacagagcagaagactctg
tgcaccgagttgaccgtaacagacatctttgctgcctccaagaacac
aactgagaaggaaaccttctgcagggctgcgactgtgctccggcagt
tctacagccaccatgagaaggacactcgctgcctgggtgcgactgca
cagcagttccacaggcacaagcagctgatccgattcctgaaacggct
cgacaggaacctctggggcctggcgggcttgaattcctgtcctgtga
aggaagccaaccagagtacgttggaaaacttcttggaaaggctaaag
acgatcatgagagagaaatattcaaagtgttcgagctga
SEQ ID NO: 11 shows the nucleotide sequence of
human IL-7.
Atgttccatgtttcttttaggtatatctttggacttcctcccctgat
ccttgttctgttgccagtagcatcatctgattgtgatattgaaggta
aagatggcaaacaatatgagagtgttctaatggtcagcatcgatcaa
ttattggacagcatgaaagaaattggtagcaattgcctgaataatga
atttaacttttttaaaagacatatctgtgatgctaataaggaaggta
tgtttttattccgtgctgctcgcaagttgaggcaatttcttaaaatg
aatagcactggtgattttgatctccacttattaaaagtttcagaagg
cacaacaatactgttgaactgcactggccaggttaaaggaagaaaac
cagctgccctgggtgaagcccaaccaacaaagagtttggaagaaaat
aaatctttaaaggaacagaaaaaactgaatgacttgtgtttcctaaa
gagactattacaagagataaaaacttgttggaataaaattttgatgg
gcactaaagaacactga
SEQ ID NO: 12 shows the nucleotide sequence of
human IL-9.
atgcttctggccatggtccttacctctgccctgctcctgtgctccgt
ggcaggccaggggtgtccaaccttggcggggatcctggacatcaact
tcctcatcaacaagatgcaggaagatccagcttccaagtgccactgc
agtgctaatgtgaccagttgtctctgtttgggcattccctctgacaa
ctgcaccagaccatgcttcagtgagagactgtctcagatgaccaata
ccaccatgcaaacaagatacccactgattttcagtcgggtgaaaaaa
tcagttgaagtactaaagaacaacaagtgtccatatttttcctgtga
acagccatgcaaccaaaccacggcaggcaacgcgctgacatttctga
agagtcttctggaaattttccagaaagaaaagatgagagggatgaga
ggcaagatatga
SEQ ID NO: 13 shows the nucleotide sequence of
human IL-15.
atgagaatttcgaaaccacatttgagaagtatttccatccagtgcta
cttgtgtttacttctaaacagtcattttctaactgaagctggcattc
atgtatcattttgggctgtttcagtgcagggcttcctaaaacagaag
ccaactgggtgaatgtaataagtgatttgaaaaaaattgaagatctt
attcaatctatgcatattgatgctactttatatacggaaagtgatgt
tcaccccagttgcaaagtaacagcaatgaagtgattctatggagtta
caagttatttcacttgagtccggagatgcaagtattcatgatacagt
agaaaatctgatcatcctagcaaacaacagtttgtcttctaatggga
atgtaacagaatctggatgcaaagaatgtgaggaactggaggaaaaa
aatattaaagaatttttgcagagttttgtacatattgtccaaatgtt
catcaacacttcttga
SEQ ID NO: 14 shows the nucleotide sequence of
human IL-21.
atgagatccagtcctggcaacatggagaggattgtcatctgtctgat
ggtcatcttcttggggacactggtccacaaatcaagctcccaaggtc
aagatcgccacatgattagaatgcgtcaacttatagatattgttgat
cagctgaaaaattatgtgaatgacttggtccctgaatttctgccagc
tccagaagatgtagagacaaactgtgagtggtcagctttttcctgct
ttcagaaggcccaactaaagtcagcaaatacaggaaacaatgaaagg
ataatcaatgtatcaattaaaaagctgaagaggaaaccaccttccac
aaatgcagggagaagacagaaacacagactaacatgcccttcatgtg
attcttatgagaaaaaaccacccaaagaattcctagaaagattcaaa
tcacttctccaaaagatgattcatcagcatctgtcctctagaacaca
cggaagtgaagattcctga
SEQ ID NO: 15 shows the nucleotide sequence of
human IL-25.
Atgagggagcgacccagattaggtgaggacagttctctcattagcct
tttcctacaggtggttgcattcttggcaatggtcatgggaacccaca
cctacagccactggcccagctgctgccccagcaaagggcaggacacc
tctgaggagctgctgaggtggagcactgtgcctgtgcctcccctaga
gcctgctaggcccaaccgccacccagagtcctgtagggccagtgaag
atggacccctcaacagcagggccatctccccctggagatatgagttg
gacagagacttgaaccggctcccccaggacctgtaccacgcccgttg
cctgtgcccgcactgcgtcagcctacagacaggctcccacatggacc
cccggggcaactcggagctgctctaccacaaccagactgtcttctac
cggcggccatgccatggcgagaagggcacccacaagggctactgcct
ggagcgcaggctgtaccgtgtttccttagcttgtgtgtgtgtgcggc
cccgtgtgatgggctag
SEQ ID NO: 16 shows the nucleotide sequence of
human IL-33, in which shown in bold is the
nucleotide sequence comprised in the propeptide.
atgaagcctaaaatgaagtattcaaccaacaaaatttccacagcaaa
gtggaagaacacagcaagcaaagccttgtgtttcaagctgggaaaat
cccaacagaaggccaaagaagtttgccccatgtactttatgaagctc
cgctctggccttatgataaaaaaggaggcctgttactttaggagaga
aaccaccaaaaggccttcactgaaaacaggtagaaagcacaaaagac
atctggtactcgctgcctgtcaacagcagtctactgtggagtgcttt
gcctttggtatatcaggggtccagaaatatactagagcacttcatga
ttcaagtatcacaggaatttcacctattacagagtatcttgcttctc
taagcacatacaatgatcaatccattacttttgctttggaggatgaa
agttatgagatatatgttgaagacttgaaaaaagatgaaaagaaaga
taaggtgttactgagttactatgagtctcaacacccctcaaatgaat
caggtgacggtgttgatggtaagatgttaatggtaaccctgagtcct
acaaaagacttctggttgcatgccaacaacaaggaacactctgtgga
gctccataagtgtgaaaaaccactgccagaccaggccttctttgtcc
ttcataatatgcactccaactgtgtttcatttgaatgcaagactgat
cctggagtgtttataggtgtaaaggataatcatcttgctctgattaa
agtagactcttctgagaatttgtgtactgaaaatatcttgtttaagc
tctctgaaacttag
SEQ ID NO: 17 shows the amino acid sequence of
the exemplary HSA-IL-7 fusion protein, in which
the linker region GGGGS is shown in underlined
and human IL-7 is fused to the C-terminal of
human serum albumin.
DAHKSEVAHRFKDLGEENFKALVLIAFAQYLQQCPFEDHVKLVNEVT
EFAKTCVADESAENCDKSLHTLFGDKLCTVATLRETYGEMADCCAKQ
EPERNECFLQHKDDNPNLPRLVRPEVDVMCTAFHDNEETFLKKYLYE
IARRHPYFYAPELLFFAKRYKAAFTECCQAADKAACLLPKLDELRDE
GKASSAKQRLKCASLQKFGERAFKAWAVARLSQRFPKAEFAEVSKLV
TDLTKVHTECCHGDLLECADDRADLAKYICENQDSISSKLKECCEKP
LLEKSHCIAEVENDEMPADLPSLAADFVESKDVCKNYAEAKDVFLGM
FLYEYARRHPDYSVVLLLRLAKTYETTLEKCCAAADPHECYAKVFDE
FKPLVEEPQNLIKQNCELFEQLGEYKFQNALLVRYTKKVPQVSTPTL
VEVSRNLGKVGSKCCKHPEAKRMPCAEDYLSVVLNQLCVLHEKTPVS
DRVTKCCTESLVNRRPCFSALEVDETYVPKEFNAETFTFHADICTLS
EKERQIKKQTALVELVKHKPKATKEQLKAVMDDFAAFVEKCCKADDK
ETCFAEEGKKLVAASQAALGLGGGGSGGGGSGGGGSDCDIEGKDGKQ
YESVLMVSIDQLLDSMKEIGSNCLNNEFNFFKRHICDANKEGMFLFR
AARKLRQFLKMNSTGDFDLHLLKVSEGTTILLNCTGQVKGRKPAALG
EAQPTKSLEENKSLKEQKKLNDLCFLKRLLQEIKTCWNKILMGTKEH
SEQ ID NO: 18 shows the amino acid sequence of
the exemplary IL-25-HSA fusion protein, in
which the linker region GGGGS is shown in
underlined and human IL-25 is fused to the
N-terminal of human serum albumin.
YSHWPSCCPSKGQDTSEELLRWSTVPVPPLEPARPNRHPESCRASED
GPLNSRAISPWRYELDRDLNRLPQDLYHARCLCPHCVSLQTGSHMDP
RGNSELLYHNQTVFYRRPCHGEKGTHKGYCLERRLYRVSLACVCVRP
RVMGGGGGSGGGGSGGGGSDAHKSEVAHRFKDLGEENFKALVLIAFA
QYLQQCPFEDHVKLVNEVTEFAKTCVADESAENCDKSLHTLFGDKLC
TVATLRETYGEMADCCAKQEPERNECFLQHKDDNPNLPRLVRPEVDV
MCTAFHDNEETFLKKYLYEIARRHPYFYAPELLFFAKRYKAAFTECC
QAADKAACLLPKLDELRDEGKASSAKQRLKCASLQKFGERAFKAWAV
ARLSQRFPKAEFAEVSKLVTDLTKVHTECCHGDLLECADDRADLAKY
ICENQDSISSKLKECCEKPLLEKSHCIAEVENDEMPADLPSLAADFV
ESKDVCKNYAEAKDVFLGMFLYEYARRHPDYSVVLLLRLAKTYETTL
EKCCAAADPHECYAKVFDEFKPLVEEPQNLIKQNCELFEQLGEYKFQ
NALLVRYTKKVPQVSTPTLVEVSRNLGKVGSKCCKHPEAKRMPCAED
YLSVVLNQLCVLHEKTPVSDRVTKCCTESLVNRRPCFSALEVDETYV
PKEFNAETFTFHADICTLSEKERQIKKQTALVELVKHKPKATKEQLK
AVMDDFAAFVEKCCKADDKETCFAEEGKKLVAASQAALGL
SEQ ID NO: 19 shows the amino acid sequence of
the exemplary IgG4mFc-IL-7 fusion protein, in
which the linker region GGGGS is shown in
underlined and human IL-7 is fused to the
C-terminal of human IgG4m Fc.
ESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMT
KNQVSLTCLVKGFYPSDIAVEWESNGQPENNYKTTPPVLDSDGSFFL
YSRLTVDKSRWQEGNVFSCSVMHEALHNHYTQKSLSLSLGKGGGGSG
GGGSGGGGSDCDIEGKDGKQYESVLMVSIDQLLDSMKEIGSNCLNNE
FNFFKRHICDANKEGMFLFRAARKLRQFLKMNSTGDFDLHLLKVSEG
TTILLNCTGQVKGRKPAALGEAQPTKSLEENKSLKEQKKLNDLCFLK
RLLQEIKTCWNKILMGTKEH
SEQ ID NO: 20 shows the amino acid sequence of
the exemplary IgG4mFc-IL-25 fusion protein, in
which the linker region GGGGS is shown in
underlined and human IL-25 is fused to the
C-terminal of human IgG4m Fc.
ESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMT
KNQVSLTCLVKGFYPSDIAVEWESNGQPENNYKTTPPVLDSDGSFFL
YSRLTVDKSRWQEGNVFSCSVMHEALHNHYTQKSLSLSLGKGGGGSG
GGGSGGGGSYSHWPSCCPSKGQDTSEELLRWSTVPVPPLEPARPNRH
PESCRASEDGPLNSRAISPWRYELDRDLNRLPQDLYHARCLCPHCVS
LQTGSHMDPRGNSELLYHNQTVFYRRPCHGEKGTHKGYCLERRLYRV
SLACVCVRPRVMG
SEQ ID NO: 21 is uPA (the substrate sequence of
urokinase-type plasminogen activator) sensitive
cleavage sequence, and ↓ shows the cleavage
site.
LSGR↓SDNH
SEQ ID NO: 22 is legumain (cysteine protease)
sensitive cleavage sequence, and ↓ shows the
cleavage site.
AAN↓L
SEQ ID NO: 23 is legumain (cysteine protease)
sensitive cleavage sequence, and ↓ shows the
cleavage site.
AAN↓V
SEQ ID NO: 24 is MMP-1 sensitive cleavage
sequence, and ↓ shows the cleavage site.
PLG↓LWA
SEQ ID NO: 25 is MMP-2/9 sensitive cleavage
sequence, and ↓ shows the cleavage site.
PAA↓LVGA
SEQ ID NO: 26 is MMP-14 sensitive cleavage
sequence, and ↓ shows the cleavage site.
SGRIGF↓LRTA
SEQ ID NO: 32 is MMP-14 sensitive cleavage
sequence, and ↓ shows the cleavage site.
PAG↓LVG
SEQ ID NO: 27 is Cathepsin B sensitive
cleavage sequence, and ↓ shows the
cleavage site.
GFLG↓
SEQ ID NO: 28 is glycin-serine linker.
(GGGGS)ₙ,
n = 1-3
SEQ ID NO: 29 is α-helix forming peptide
linker.
(EAAAK)ₙ,
n = 2-5
SEQ ID NO: 30 shows the amino acid sequence of
the exemplary IL-25 + IL7 recombinant protein,
in which the sequence of the uPA cleavable
peptide linker between two active components is
shown in bold, uPA recognition substrate
sequence is shown in single-underlined, and
tumor penetrating peptide iRGD is shown in
double-underlined.
YSHWPSCCPSKGQDTSEELLRWSTVPVPPLEPARPNRHPESCRASED
GPLNSRAISPWRYELDRDLNRLPQDLYHARCLCPHCVSLQTGSHMDP
RGNSELLYHNQTVFYRRPCHGEKGTHKGYCLERRLYRVSLACVCVRP
RVMGGGSGLSGRSDNHGGGGSDCDIEGKDGKQYESVLMVSIDQLLDS
MKEIGSNCLNNEFNFFKRHICDANKEGMFLFRAARKLRQFLKMNSTG
DFDLHLLKVSEGTTILLNCTGQVKGRKPAALGEAQPTKSLEENKSLK
EQKKLNDLCFLKRLLQEIKTCWNKILMGTKEHGGGGSGGGGSGGGGS

[symbol]

SEQ ID NO: 31 shows the amino acid sequence of
the exemplary IL-25 + IL-7 + IL-2 recombinant
protein, in which the sequence of the uPA
cleavable peptide linker between active
components is shown in bold, uPA substrate
sequence is shown in single-underlined, and
tumor penetrating peptide iRGD is shown in
double-underlined.
YSHWPSCCPSKGQDTSEELLRWSTVPVPPLEPARPNRHPESCRASED
GPLNSRAISPWRYELDRDLNRLPQDLYHARCLCPHCVSLQTGSHMDP
RGNSELLYHNQTVFYRRPCHGEKGTHKGYCLERRLYRVSLACVCVRP
RVMGGGSGLSGRSDNHGGGGSDCDIEGKDGKQYESVLMVSIDQLLDS
MKEIGSNCLNNEFNFFKRHICDANKEGMFLFRAARKLRQFLKMNSTG
DFDLHLLKVSEGTTILLNCTGQVKGRKPAALGEAQPTKSLEENKSLK
EQKKLNDLCFLKRLLQEIKTCWNKILMGTKEHGGGSGLSGRSDNHGG
GGSAPTSSSTKKTQLQLEHLLLDLQMILNGINNYKNPKLTAMLTKKF
YMPKKATELKHLQCLEEELKPLEEVLNLAQSKNFHLRPRDLISNINV
IVLELKGSETTFMCEYADETATIVEFLNRWITFCQSIISTLTGGGGS
GGGGSGGGGS

[symbol]

### DETAILED DESCRIPTION OF EMBODIMENTS

Unless otherwise stated, each term in the present disclosure has the same meaning as commonly understood by one of ordinary skill in the art.

As used herein, the terms “γc-cytokine” or “γc-cytokine family” and “common cytokine receptor gamma-chain family” are used interchangeably and refer to the family of cytokines composed of six members (i.e., IL-2, IL-4, IL-7, IL-9, IL-15 and IL-21), all members of which signal through a receptor complex containing a common cytokine receptor gamma chain subunit.

In one embodiment, typical suitable γc-cytokines include the mature portions in which the respective signal peptide sequences are removed or the active portions in which internal partial amino acids are deleted. For example, IL-7 includes but is not limited to the mature IL-7 polypeptide portion (the signal peptide sequence in the sequence as set forth in SEQ ID NO: 3 is removed), or includes sequences in which some of the amino acids at positions 96 to 114 of SEQ ID NO: 3 are deleted.

As used herein, “IL-25”, also known as IL-17E, is a member of the structurally related IL-17 family but is functionally distinct from other IL-17 family members. A typical suitable IL-25 includes, but is not limited to, the mature IL-25 polypeptide portion (the signal peptide sequence in the sequence as set forth in SEQ ID NO: 7 is removed).

As used herein, “IL-33” is a new member of the IL-1 family. A typical suitable IL-33 includes, but is not limited to, the mature portion of the IL-33 polypeptide (the bold sequence in the sequence as set forth in SEQ ID NO: 16 is removed).

In the present disclosure, reference to the amino acid sequences of various cytokines and the nucleotide sequences encoding the same encompasses their corresponding variants or mutants. Various variants or mutants of cytokines known in the art can be used herein, and can be found at http://www.ncbi.nlm.nih.gov/. Generally, a variant of a particular nucleotide sequence will have at least about 70%, 75%, 80%, 85%, 86%, 87%, 88%, 89%, 90%, 91%, 92%, 93%, 94%, 95%, 96%, 97%, 98%, 99%, 99.5% or 99.9% or higher sequence identity with the particular nucleotide sequence, or be complementary sequences of the above. Such variant sequences include additions, deletions or substitutions of one or more nucleic acid residues, which may result in the addition, removal or substitution of corresponding amino acid residues. Sequence identity is determined by sequence alignment programs known in the art, including hybridization techniques.

Thus, nucleotide sequence variants can differ from the indicated sequences by as few as 1-15 nucleotides, as few as 1-10 (e.g., 6-10), as few as 5, as few as 4, 3, 2 or even 1 nucleotide. Accordingly, amino acid sequence variants can differ from the indicated sequences by as few as 1-15 amino acids, as few as 1-10 (eg, 6-10), as few as 5, as few as 4, 3, 2 or even 1 amino acid.

In one embodiment, taking IL-7 as an example, its variants may be active variants having at least 70%, 75%, 80%, 85%, 86%, 87%, 88%, 89%, 90%, 91%, 92%, 93%, 94%, 95%, 96%, 97%, 98%, 99%, 99.5% or 99.9% or higher identity with the amino acid sequence as set forth in SEQ ID NO:3. Correspondingly, the variants of the nucleic acid encoding IL-7 may be those having at least 70%, 75%, 80%, 85%, 86%, 87%, 88%, 89%, 90%, 91%, 92%, 93%, 94%, 95%, 96%, 97%, 98%, 99%, 99.5% or 99.9% or higher identity with the nucleotide sequence as set forth in SEQ ID NO: 11.

In certain embodiments, to extend the in vivo half-life of a peptide or proteinaceous drug, and to improve its pharmacokinetic and pharmacodynamic profile, the various cytokines provided herein may also have any suitable chemical modifications known in the art, e.g., PEGylation, Xtenylation, PASylation, Hesylation or fusion with other proteins. The various cytokines provided herein may also have or have no a tag, including but not limited to any subtypes or variants of human antibody Fc, human serum albumin (HSA) or human transferrin, and these tags are fused to the N-terminal or C- terminal of cytokines.

Specifically, Xtenylation involves an XTEN technology recently developed by Amunix, Inc., which relies on the peptide chains of different lengths without specific conformation consisting of six amino acids, Ala, Glu, Gly, Pro, Ser, and Thr, termed as XTEN sequences. When linked to the peptide or proteinaceous drug via a gene fusion method, it increases the solubility and stability of the drug, and is characterized by low immunogenicity, high product purity, and complete biodegradation. In addition, XL-protein Inc. uses a random coiled protein structure constructed by repeated Pro, Ala, and Ser. This technique, called PASylation, uses these three amino acids to form a PAS sequence of 200-600 amino acid residues. Linking the PAS sequence to proteinaceous or peptide drugs via a gene fusion method, increases the volume, thereby preventing rapid filtration of the kidney and prolonging the half-life.

In one embodiment, with the IL-7 and IL-25 as an example, the amino acid sequences of their human serum albumin (HSA) fusion proteins are shown in SEQ ID NOs: 17 and 18, respectively, and the amino acid sequences of the IgG4mFc fusion proteins are shown in SEQ ID NOs: 19 and 20, respectively.

In certain embodiments, in order to enhance the enrichment of a protein of interest in a tumor, a tumor-penetrating peptide can be fused to the N-terminal or C-terminal of the cytokines, including but not limited to fusion of cRGD peptide or cNGR at its N-terminal, fusion of iRGD peptide or iNGR peptide or pHLIPs peptide at its C-terminal; a tumor-targeted single-chain or single-domain antibody can be fused to the N-terminal or C-terminal of cytokines, including but not limited to fusion with single-chain or single-domain antibody targeting Tn-Mucl, EDB-Fibronectin, mesothelin, FAP, CEA, HER2, GD2, PD-L1 or EGFR.

In certain embodiments, in order to simplify the dosage form, process, and the convenience of application of a combination drug, the various combinations of cytokines provided herein can be achieved by fusion proteins, and the peptide linker which are cleavable in vivo or intratumorally can be comprised between the active components of the recombinant protein.

In one embodiment, with the IL-7 and IL-25 as an example, the amino acid sequence of the recombinant protein of IL-25 and IL-7 comprising the uPA cleavable linker and the tumor penetrating peptide iRGD is set forth in SEQ ID NO: 30.

In one embodiment, with the IL-7, IL-25 and IL-2v variants (R38A/F42K) as an example, the amino acid sequence of the recombinant protein of IL-25, IL-7 and IL-2v comprising the uPA cleavable linker and the tumor penetrating peptide iRGD is set forth in SEQ ID NO:31.

As used herein, the term “recombinant protein” refers to a specific recombinant protein molecule which is expressed by the use of genetic recombination techniques known in the art to obtain a recombinant vector linked to a gene fragment that can be translated into a protein of interest, which is then transformed into a host cell which expresses the protein of interest.

In one embodiment, the recombinant protein provided herein includes, but is not limited to, one or more selected from the group consisting of γc-cytokine family members, such as IL-2, IL-4, IL-7, IL-9, IL-15 or IL-21, and IL-25 or IL-33.

In another embodiment, the referred nucleic acid coding sequences of these cytokines herein may be present in separate vectors; or the nucleic acid coding sequences of these cytokines are present in the same vector in combinations of any two or three.

Further, the recombinant protein provided herein can be expressed by different cells or populations of cells, or by the same cell or population of cells.

Alternatively, the recombinant nucleic acid molecule or vector provided herein can be included in different cells or population of cells, or can be included in the same cell or population of cells.

As used herein, the term “vector” refers to any recombinant polynucleotide that can be used to introduce heterologous DNA into a host cell to result in phenotype transformation. One type of vector is a plasmid that involves a circular double-stranded nucleic acid into which other deoxyribonucleic acid fragments can be inserted. Another type of vector is a viral vector in which an exogenous DNA fragment can be inserted into a viral genome, and transcripts can be packaged into infectious viral particles and transduced into target cells. Upon transduction, some viral vectors, such as retroviral or lentiviral vectors, can be integrated into the host cell genome and replicate together with the host genome.

Furthermore, vectors capable of directing gene expression are referred to as “expression vectors”. In particular, an expression vector is capable of replicating and expressing a gene of interest in a transformed or transfected host cell. The expression vector can include one or more phenotypic selection markers and a replication origin sequence to ensure amplification of the maintained vector within organism's cells. Expression vectors also include the expression of a promoter-driven polypeptide in a cell. A suitable expression vector can be a plasmid or a viral vector.

As used herein, the term “prevention and/or treatment” includes preventing, inhibiting, curing, alleviating or ameliorating a malignant tumor, as well as preventing or delaying the metastasis of primary cancer.

In certain embodiments, the malignant tumors that are prevented and/or treated include malignant tumors at various stages, such as sarcomas and carcinomas, including but not limited to the following types: fibrosarcoma, mucinous sarcoma, liposarcoma, chondrosarcoma, osteosarcoma, chordoma, angiosarcoma, endothelial sarcoma, lymphangiosarcoma, lymphangial endothelial sarcoma, synovial sarcoma, mesothelioma, Ewing's tumor, leiomyosarcoma, rhabdomyosarcoma, colon cancer, pancreatic cancer, breast cancer, ovarian cancer, prostate cancer, squamous cell carcinoma, basal cell carcinoma, adenocarcinoma, sweat gland cancer, sebaceous gland cancer, papillary carcinoma, papillary adenocarcinoma, bronchial carcinoma, medullary carcinoma, renal cell carcinoma, liver cancer, cholangiocarcinoma, choriocarcinoma, seminoma, embryonic carcinoma, nephroblastoma, cervical cancer, testicular tumor, lung cancer, small cell lung cancer, bladder cancer, epithelial cancer, glioma, astrocytoma, medulloblastoma, craniopharyngioma, ependymoma, pineal tumors, hemangioblastoma, acoustic neuroma, meningioma, oligodendroglioma, melanoma, neuroblastoma, retinoblastoma.

In one embodiment, the sarcoma that is prevented and/or treated is such as fibrosarcoma, mucinous sarcoma, liposarcoma, chondrosarcoma, osteosarcoma, chordoma, angiosarcoma, endothelial sarcoma, lymphangiosarcoma, lymphangial endothelial sarcoma, synovial sarcoma, mesothelioma, Ewing's tumor, leiomyosarcoma, rhabdomyosarcoma, etc.

In another embodiment, the cancer that is prevented and/or treated is such as colon cancer, pancreatic cancer, breast cancer, ovarian cancer, prostate cancer, squamous cell carcinoma, basal cell carcinoma, adenocarcinoma, sweat gland cancer, sebaceous gland cancer, papillary carcinoma, papillary adenocarcinoma, bronchial carcinoma, medullary carcinoma, renal cell carcinoma, liver cancer, cholangiocarcinoma, choriocarcinoma, seminoma, embryonal carcinoma, nephroblastoma, cervical cancer, testicular tumor, lung cancer, small cell lung cancer, bladder cancer, epithelial cancer, glioma, astrocytoma, medulloblastoma, craniopharyngioma, ependymoma, pineal tumor, hemangioblastoma, acoustic neuroma, meningioma, oligodendroglioma, melanoma, neuroblastoma, retinoblastoma, etc.

In yet another embodiment, the advanced cancer that is prevented and/or treated includes malignant melanoma, lung cancer, colon cancer, colon cancer, liver cancer, head and neck cancer, sarcoma, prostate cancer, and advanced cancer which has developed to a certain stage and is unable to be cured by conventional therapy.

As used herein, the term “combination therapy” refers to the administration of the combination of at least one of γc-cytokine or an active part or variant thereof and at least one of IL-25 or IL-33 or an active part or variant thereof to a subject in need thereof. Combination therapy can provide “synergistic” and “synergistic effects”, i.e., the effect achieved when the active ingredients are used together is higher than the sum of the effects achieved when the compounds are used separately. When the active ingredients are: (1) co-prepared and administered, or simultaneously delivered in the form of a combined preparation; (2) as separate preparations, delivered alternately or in parallel; or (3) by some other means, synergistic effects can be achieved.

For example, in one embodiment, the combinations provided herein comprise or consist of: i) any one, two, three, four, five or six of interleukins selected from the group consisting of IL-2, IL-4, IL-7, IL-9, IL-15 and IL-21; and ii) any one or two of interleukins selected from the group consisting of IL-25 and IL-33.

In a specific embodiment, the combinations provided herein comprise or consist of: i) any one or two of interleukins selected from the group consisting of IL-2, IL-4, IL-7, IL-9, IL-15 and IL-21; and ii) any one of interleukins selected from the group consisting of IL-25 and IL-33.

In another embodiment, the combinations provided herein comprise or consist of: i) any one or two interleukins selected from the group consisting of IL-7, IL-9 and IL-15; and ii) any one of interleukins selected from the group consisting of IL-25 and IL-33.

In still another embodiment, the combinations provided herein comprise or consist of: i) any one or two of interleukins selected from the group consisting of IL-7, IL-9 and IL-15; and ii) IL -25.

Further, in one embodiment, the combinations provided herein comprise or consist of: i) IL-7; and ii) any one of interleukins selected from the group consisting of IL-25 and IL-33.

Still further, in another embodiment, the combinations provided herein comprise or consist of: i) IL-7; ii) IL-25; and iii) any one of interleukins selected from the group consisting of IL-2, IL-9 and IL-15.

In certain embodiments, a combination comprising three interleukins exhibits more potent inhibitory and less time-consuming effect on tumors as compared to a combination comprising two interleukins.

In particular embodiments, the interleukin combinations provided herein can be administered to an individual simultaneously or sequentially.

In a specific embodiment, the interleukin combination provided herein can be administered intravenously or subcutaneously or intraperitoneally or intramuscularly to an individual.

As used herein, the term “therapeutically effective amount” can be determined on a case-by-case basis, and can be readily grasped by one of ordinary skill in the art based on the actual amount of drug required, such as being determined based on the patient's weight, age, and condition. Since extracts are all non-toxic ingredients, they can be administered directly as desired, in which the composition does not contain a pharmaceutically acceptable carrier. When a composition contains a pharmaceutically acceptable carrier, they can be mixed in a conventional manner in the pharmaceutical field to prepare a desired drug.

Hereinafter, specific embodiments of the combination therapy herein will be described by taking a combination of IL-7 and IL-25 as an example.

In one embodiment, a combination of IL-7 and IL-25 or a combination of active mutants thereof is administered to a mammalian host. In another embodiment, in addition to the purified recombinant proteins, IL-7 and IL-25 may also be derived from genetically engineered cells, including but not limited to any transformed cells, autologous cells such as dendritic cells or T cells, which can be transformed by viral vectors to express active IL-7 or IL-25 or both, and can be applied to an organism. In yet another embodiment, the transformed cells or cell lines can be encapsulated in a hydrogel such as a sodium alginate gel, thereby being applied to an organism.

In one embodiment, the preferred dose of the IL-7 and IL-25 recombinant protein for individual treatment is 0.0001-10 mg/kg body weight/day. In another embodiment, upon microencapsulation of an engineered cell line expressing IL-7 or IL-25, the preferred dose for an organism is between 1×10⁶ and 1×10¹².

In certain embodiments, the combination of the cytokines IL-7 and IL-25 as described herein can be obtained by mixing IL-7- and IL-25-producing cells. In a specific embodiment, microencapsulated engineered cells that simultaneously produce both cytokines of IL-7 and IL-25 can be injected subcutaneously or intraperitoneally into a host.

In certain embodiments, the combination of the cytokines IL-7 and IL-25 as described herein can be delivered by injecting a recombinant fusion protein comprising an intratumorally cleavable peptide between the IL-7 and IL-25 molecules. In a specific embodiment, the recombinant protein can be injected intravenously or intraperitoneally or intratumorally into a host.

When engineered autologous dendritic cells capable of simultaneously secreting both

IL-7 and IL-25 are used as a combination therapy, the preferred dose is between 1×10⁶ and 1×10¹² cells, and these cells can be injected subcutaneously or intramuscularly into a host. Similarly, if the cells used are T cells, the same dose can be used, but T cells are injected intravenously into a host.

The following examples are only for illustrative purposes and not intended to limit the scope of the application. Unless otherwise indicated, the examples are performed according to routine experimental conditions, such as the Sambrook J & Russell D W, Molecular cloning: a laboratory manual, 2001, or those as suggested by the manufacturer's instructions.

### EXAMPLES

### Example 1

In this example, IL-7 is taken as an example to provide a method for preparing a tumor cell pool for producing cytokines, which specifically comprises the following steps:

In the first step, the cDNA of the mouse cytokine IL-7 containing the coding sequence and the signal peptide was synthesized from a self-synthesized mouse spleen and lung reverse transcription reaction products (manufactured by the reverse transcription reaction kit of Thermo Scientific Inc.) by PCR.

In the second step, these genes were separately cloned into the retroviral vector pBabe-SV40-puro (purchased from http://www.addgene.org/) and the resultant sequences were confirmed.

In the third step, human embryonic kidney HEK293T cells (purchased from http://www.atcc.org/) were co-transfected with pCL-Ampho helper plasmid (purchased from NOVUS Biologicals Inc.) and pBabe-derived plasmid expressing cytokines to produce a retrovirus.

Briefly, the medium of HEK293T cells plated in p100 cell culture dishes on the previous day was replaced with pre-warmed pen/strp-free DMEM-10 medium (DMEM containing 10% fetal calf serum and glutamine) 10 min prior to transient transfection. 5 μg of pCL-Ampho helper plasmid and 5 μg of pBabe-derived plasmid were mixed in an Eppendof tube, and then added 1 ml of Opti-MEM medium and 30 μl of PEI transfection reagent. After incubation for 10 minutes at room temperature, the DNA pellet-containing mixture was added to the culture dish of the above HEK-293T cells. After 6 hours, Pen/strp was added overnight, and then replaced with new DMEM-10 medium. After 24 hours, the supernatant containing the retrovirus was collected and sterilized through a 45 μM filter.

In the fourth step, mouse melanoma B16F10 cells (purchased from http://www.atcc.org/) were transduced with retrovirus, and a tumor cell pool producing cytokine IL-7 was obtained after enrichment.

Briefly, B16F10 cells cultured in p100 cell culture dishes were transduced overnight with the resultant retrovirus plus polybrene (final concentration of 8 μg/mL), and then the culture medium was changed to DMEM10 medium and puromycin (final concentration of 3 μg/mL) was added to enrich cells transduced by viruses. The cells were expanded and frozen in a cryopreservation solution for long-term storage.

Similarly, tumor cell pools comprising other cytokines, such as IL-1b, IL-2, IL-4, IL-5, IL-8, IL-9, IL-13, IL-15, IL-17A, IL-18, IL-21, IL-25, IL-28, IL-36, can be produced. Furthermore, the above two or more cell pools can be pooled to produce a tumor cell pool that co-expresses two or more cytokines.

### Example 2

Upon the preparation of a tumor cell pool for producing cytokines, in this example, combination of cytokines which synergistically inhibits tumor growth is further screened by in vivo experiments.

Experimental animals: Female C57BL/6 mice, approximately 2-3 months old, were shaved on both sides to facilitate injection to tumor cells and measurement of tumor size.

Cell preparation: B16F10 primary cells that did not grow to complete confluence were trypsinized, and the cell concentration was determined by a hemocytometer.

In vivo experiments: 5×10⁵ B16F10 primary cells suspended in RPMI-1640 medium were injected intradermally (i.d.) into the left of female C57BL/6 mice as response tumors. Similarly, a tumor cell pool expressing specific cytokines or combinations thereof (each 1×10⁶ cells) were injected intradermally into the right as cytokine tumors. Mice were sacrificed on day 16 and tumor size was measured.

“cytokine tumor” used herein is a drug-sustaining generator that will continuously release cytokines to activate the body's immune system. The activated immune system will kill the cytokine tumor itself and response tumors, while the size of “effect tumors” reflects the actual therapeutic effect of the combination drug. The drug combination for the inhibition of unilateral tumors (mostly for cytokine tumors themselves) indicates that the combination requires a relatively high concentration of cytokines or has the effect of stimulating immune cells resident in local tissues to inhibit tumors, or the combination has strong chemotaxis-promoting effect and can be used as a control. A combination exhibiting significant inhibitory effect on both sides of tumors is considered to be a therapeutically valuable combination.

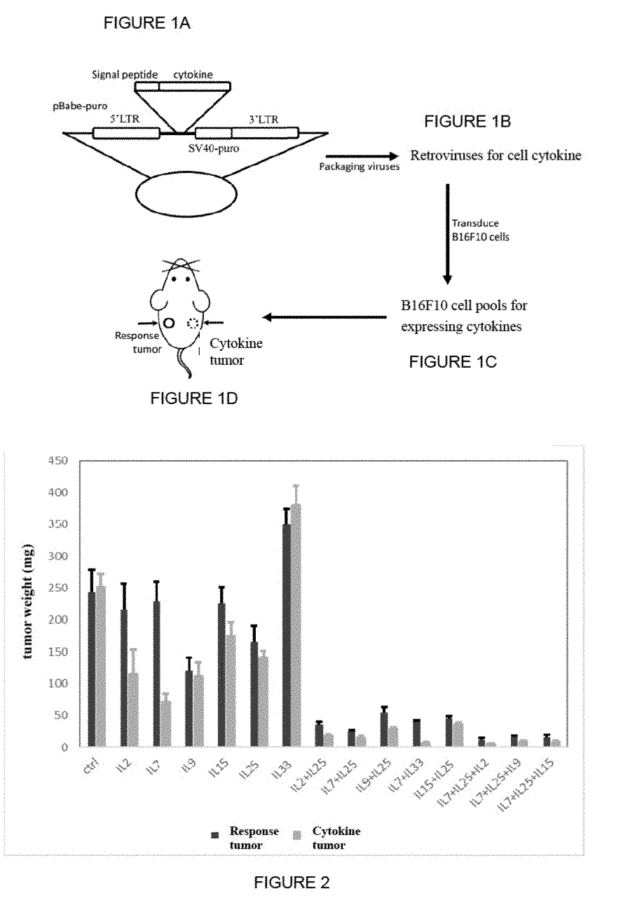

The experimental flow diagram of the above-described preparation of a cytokine-producing tumor cell pool and verification of tumor growth inhibition is shown in FIG. 1.

FIG. 2 shows exemplary results demonstrating tumor growth inhibition using different cytokine-producing tumor cell pools or combinations thereof.

### Example 3

This example utilizes a pool of tumor cells producing the cytokines IL-7 and IL-25 to treat tumors, as follows.

Female C57BL/6 mice of 2-3 month old were divided into the following 4 groups with 10 per group:

Control group, 5×10⁵ B16F10 primary cells were injected intradermally on both sides;

IL-7 treatment group, the IL-7-producing cell pool (1 million) was injected into the right side of the mouse, and the B16F10 primary cell was injected into the left side (5×10⁵ as an response tumor);

IL-25 treatment group, the IL-25-producing cell pool was intradermally injected into the right side, and the B16F10 primary cells were intradermally injected into the left side;

IL-7+IL-25 treatment group, 1 million of each IL-7- and IL-25-producing cell pool were intradermally injected into the right side, and the B16F10 primary cells were intradermally injected into the left side.

After 4 days, the tumor area on both sides was measured daily using a vernier caliper.

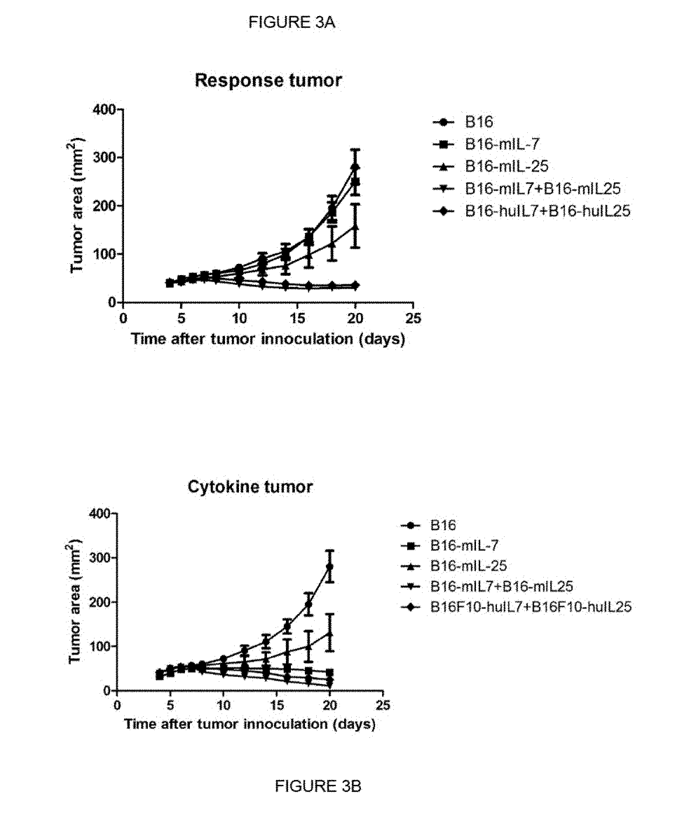

The experimental results are shown in FIG. 3.

In addition, the cell pool co-expressing hu-IL-7 and hu-IL-25 obtained the same result.

### Example 4

This example provides expression and purification of the recombinant hu-IL-7 and hu-IL-25 proteins, and the therapeutic effects thereof on tumors.

1. Expression and Purification of Recombinant hu-IL-7 or hu-IL-25 Protein

The 6× His N-terminally labeled human IL-7 or IL-25 cDNA non-signal peptide region coding sequence was synthesized by PCR and cloned into the pET15 plasmid vector (purchased from Novagen Inc.).

After sequence verification, the vector was transformed into E. coli BL21 (DE3) (purchased from NEB Inc.), and the transformed single colonies were inoculated into 10 ml of LB medium (containing 100 μg/ml ampicillin) and cultured overnight on a shaker at 250 rpm/min at 37° C. 5 ml of the initial culture was transferred to 500 ml of ampicillin-containing LB medium, and cultured on a shaker at 25° C. until the OD600 reached about 0.4. IPTG was added to 200 μM to induce culture overnight.

The obtained culture was centrifuged and resuspended in suspension buffer (containing 20 mM HEPES pH 7.5, sodium chloride 50 mM, 0.5 mM EDTA, a protease inhibitor). The cells were disrupted by ultrasonic wave, and the supernatant was removed after high speed centrifugation. The precipitate containing the inclusion body was washed once with a suspension buffer, and then dissolved in a denaturing buffer (50 mM Tris-HCl pH 7.5, 500 mM NaCl, 6 M guanidine hydrochloride). Upon centrifugation at high speed, the supernatant contained denatured inclusion body proteins.

The resulted supernatant was passed through a 1.5 ml Ni-NTA agarose affinity chromatography column to further purify the denatured proteins. The column was washed with 20 ml of denaturing buffer, and then washed with 10 ml of denaturing buffer containing 10 mM imidazole. The recombinant protein was eluted with 10 ml of denaturing buffer containing 0.1 M imidazole.

The protein renaturation procedure was subsequently performed. The eluted mixed proteins were dialyzed with dialysis buffer A (50 mM Tris-HCl pH8.0, sodium chloride 50 mM, 1 mM EDTA, 10 mM DTT, 6 M guanidine hydrochloride) for 24 hours at 4° C. They were then dialyzed overnight with dialysis buffer A without DTT. The proteins were adjusted to 0.2 mg/ml of concentration, and then dialyzed with renaturation buffer (50 mM Tris-HCl pH 8.0, 100 mM arginine, 5 mM reduced glutathione, 0.5 mM oxidized glutathione, 50 mM NaCl) for 3 days at 4 ° C. with dialysate changing daily. The proteins were then dialyzed overnight with glutathione-free renaturation buffer, and finally dialyzed with 1× PBS for one day. The renatured protein solution was centrifuged to remove residual aggregates. Residual trace amount of LPS was removed through a 1 ml polymyxin B agarose column. The proteins were concentrated by ultrafiltration. The purified protein was subpackaged and stored.

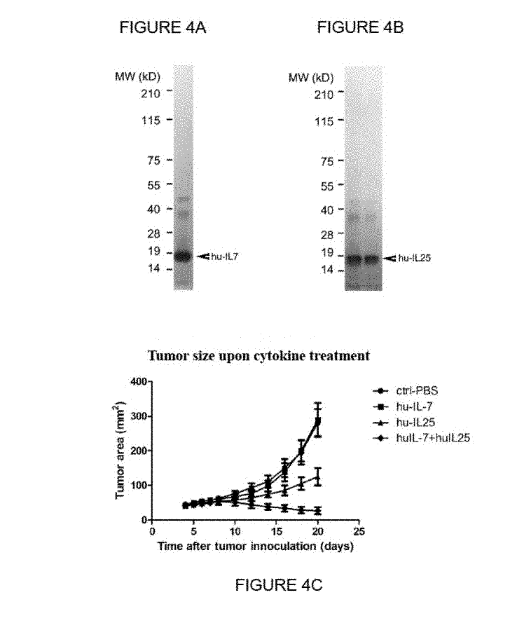

The protein concentration was determined by the Bradford method. 10 μg of the partially purified protein was subjected to 4-20% SDS PAG gradient gel electrophoresis, and stained with Coomassie brilliant blue. The results are shown in FIG. 4A and FIG. 4B.

2. Treatment of Tumors with the Combination of Purified IL-7 and IL-25 Proteins

Tumors were treated with the combination of purified IL-7 and IL-25 proteins. If the hosts had used antibiotics, the administration of all antibiotics should be terminated at least one of week prior to treatment and during treatment, as antibiotic administration will severely affect the efficacy of combination therapy or make treatment completely fail. C57BL/6 mice were divided into 4 groups with 10 per group. All mice were intradermally injected with 5×10⁵ B16F10 cells on both sides. After one day of injection, the mice were treated as follows:

Control group, 0.2 ml PBS;

hu-IL-7 protein treatment group, 0.2 ml PBS containing 5 μg hu-IL-7 protein;

hu-IL-25 protein treatment group, 0.2 ml PBS containing 5 μg hu-IL-25 protein;

hu-IL-7 and hu-IL-25 protein combination treatment group, 0.2 ml PBS containing 5 μg of each hu-IL-7 protein and hu-IL-25 protein.

The mice were intraperitoneally injected twice a day, and the tumor area was measured daily using a vernier caliper four days after the start of treatment.

The results are shown in FIG. 4C.

### Example 5

This example utilizes the combination of purified hu-IL-7-HSA and hu-IL-25-HSA to treat human tumors.

Recombinant HSA-IL-7 (as shown in SEQ ID NO: 3) and IL-25-HSA (as shown in SEQ ID NO: 4) were respectively expressed in CHO cells (Chinese hamster ovary cells), and these two purified recombinant proteins were produced in GMP-compliant environment for human tumor therapy.

Administration of all antibiotics was terminated at least one of week prior to treatment and during treatment. Hu-IL-7-HSA and hu-IL-25-HSA were mixed in proportion and then administered intravenously or intraperitoneally or subcutaneously or intramuscularly. The therapeutic dose is 0.0001-100 mg/kg body weight and it was administered once at intervals of average 5-30 days. The tumor size was measured four months later to determine the therapeutic effect.

### Example 6

This example utilizes the combination of purified IgG4m-Fc-IL-7 and IgG4m-Fc-IL-25 to treat human tumors.

Recombinant human IgG4m-Fc-IL-7 (as shown in SEQ ID NO: 5) and IgG4m-Fc-IL-25 (as shown in SEQ ID NO: 6) were respectively expressed in human retinal PER.C6 cells, and these two purified recombinant proteins were produced in GMP-compliant environment for human tumor therapy.

Administration of all antibiotics was terminated at least one of week prior to treatment and during treatment. IgG4m-Fc-IL-7 and IgG4m-Fc-IL-25 were mixed in proportion and then administered intravenously or intraperitoneally or subcutaneously or intramuscularly. The therapeutic dose was 0.0001-10 mg/kg body weight, and it was administered once at intervals of average 5-30 days. The tumor size was measured four months later to determine the therapeutic effect.

### Example 7

This example utilizes autologous T cells co-expressing IL-7 and IL-25 to treat tumors.

The expression cassette co-expressing hu-IL-7, hu-IL-25, and IRES-hu-CD19 with the intracellular domain removed, was cloned into the transposon vector pSBbi bidirectional promoter vector (purchased from Addgene).

50 ml of peripheral blood from a donor was taken, and lymphocytes were isolated by Ficoll-paque. After immunofluorescence staining, CD3⁺PD1⁺CD25⁻ T cells were sorted by flow cytometry.

In addition, T cell populations also can be isolated from Tumor infiltrating lymphocytes isolated from fresh tumor tissues.

Transposon vector (2.5 μg) co-expressing hu-IL-7 and hu-IL-25-IRES-hu-CD19 and SB100× (0.5 μg) transposase expression vector were co-transfected into prepared 1×10⁷ T cells by electroporation (Lonza, T cell nucleofection kit). After overnight incubation, cells were incubated with mouse anti-human CD3 antibody-conjugated magnetic beads and mouse anti-human CD28 antibody for 24 to 48 hours, and then incubated with hu-IL-2-containing medium for two days. The transfected T cells were sorted by Biotin-anti-hu-CD19 antibody and magnetic beads of streptavidin and continued to expand to the desired dose.

The prepared T cells were intravenously returned to the donor at a dose of 10⁵-10⁹ cells/kg body weight/time. The T cells was returned once every 5 to 100 days, and the tumor size was measured four months later to determine the therapeutic effect.

### Example 8

This example utilizes cells co-expressing IL-7 and IL-25 or IL-33 encapsulated into sodium alginate microcapsules to treat tumors.

Cells that co-express IL-7 and IL-25 or IL-7 and IL-33 were respectively encapsulated into a multi-layer sodium alginate microencapsulation system (Bhuhbal et al., 2014, Sci. Rep. 4: 6856) as follows.

Lentiviral vectors co-expressing IL-7 and IL-25 or IL-7 and IL-33 were transduced into baby hamster kidney cells (BHK) and cloned and expanded.

The cells were precipitated by centrifugation (final concentration of 6 million/ml) and mixed with medium guluronic acid (G)-sodium alginate (final concentration of 3.4%), and converted into droplets by a 27 G needle using an electrostatic bead generator. The droplets were collected in 100 mM CaCl₂ gel solution for 5 minutes. These microspheres were incubated with 0.05% polylysine (PLL). The PLL-coated microcapsules were then co-incubated with 0.34% of medium-G sodium alginate dissolved in calcium-free Krebs-Ringer-Hepes (KRH) solution (120 mM NaCl, KCl 5 mM, 1 mM magnesium chloride, 25 mM sodium bicarbonate, 5.5 mM Hepes, 1 mM glucose, pH 7.2±0.15) to form sodium alginate-poly-L-lysine-sodium alginate (APA) microcapsules. The cells containing the medium G alginate-PLL membrane were then suspended in 2% high G sodium alginate solution. This solution was collected in a 100 mM CaCl₂ gel solution through a 23 G needle using another type of droplet generator. These microencapsulated cells were cultured in a cell culture flask with culture medium (DMEM, 10% (v/v) fetal bovine serum, antibiotics and antifungal agent) in a standard tissue culture incubator. The medium was changed every other day. Administration of all antibiotics to the host was terminated at least one of week prior to treatment and during treatment. The microencapsulated cells were then implanted intradermally or intraperitoneally into the animal body at a dose between 1×10⁷ and 1×10¹² cells, once every 2-12 months, as a combination for cancer therapy.

### Example 9

This example utilizes adenovirus co-expressing hu-IL-7 and hu-IL-25 to treat tumors.

Adenovirus was generated using the adenovirus pAdEasy system (Luo et al 2007, Nat. Protocol, 2, 12360-1247) as follows.

The hu-IL-7 and hu-IL-25 coding sequences were cloned into the shuttle vector of pAdTrack-CMV. After identification by sequencing, the vector was linearized with PmeI, and 0.5 μg of linearized vector was transformed into pAdEasy competent cells by electroporation. The identified cloned plasmid was then transformed into DH10B competent cells, and the recombinant adenovirus plasmid was amplified and purified. 3 μg of adenoviral plasmid was linearized with PacI and transfected into HEK293 adenovirus packaging cells. After 14-20 days, the cells were collected and the adenovirus was extracted by repeated freeze-thaw method. The high titer adenovirus was then amplified and purified stepwise. The titer of the resultant adenovirus was measured and the amount of IL-7 and IL-25 produced by the infected target cells in vitro was measured by ELISA. The quality-controlled adenovirus was injected into the patient's tumor with a syringe, and the dose was determined according to the size of the tumor. 10⁸-10¹³ pfu of adenoviruses were injected into each tumor.

### Example 10

This example tests the therapeutic activity against tumors using purified hu-IL-25-hu-IL-7-iRGD recombinant protein.

The recombinant hu-IL-25-hu-IL-7-iRGD recombinant protein (as shown in SEQ ID NO: 30) was expressed in CHO cells (Chinese hamster ovary cells), and the recombinant protein was produced and purified in GMP-compliant environment for tumor therapy.

Administration of all antibiotics was terminated at least one of week prior to treatment and during treatment. The recombinant hu-IL-25-hu-IL-7-iRGD recombinant protein was administered intravenously or intraperitoneally or subcutaneously or intramuscularly or intratumorally, at a therapeutic dose of 0.0001 to 10 mg/kg body weight at average 1-4 times per day, or continuously administered by an insulin pump, and the tumor size was measured four months later to judge the therapeutic effect.

1.-26. (canceled)

27. A pharmaceutical combination for preventing and/or treating a malignant tumor, comprising:
i) at least one of γc-cytokine or an active part or variant thereof, or a vector or a cell or a population of cells capable of producing at least one of γc-cytokine or an active part or variant thereof, or a nucleic acid molecule capable of encoding one of γc-cytokine or an active portion or a variant thereof; and
ii) at least one of IL-25 or IL-33 or an active part or variant thereof, or a vector or a cell or a population of cells capable of producing at least one of IL-25 or IL-33 or an active part or variant thereof, or a nucleic acid molecule capable of encoding at least one of IL-25 or IL-33 or an active part or variant thereof;
wherein the at least one of γc-cytokine is selected from the group consisting of IL-2, IL-7, IL-9 and IL-15.

28. The pharmaceutical combination according to claim 27, wherein the pharmaceutical combination is selected from the group consisting of:
i) IL-7 and/or IL-9 or an active part or variant thereof, or a vector or a cell or a population of cells capable of producing IL-7 and/or IL-9 or an active part or variant thereof, or a nucleic acid molecule capable of encoding IL-7 and/or IL-9 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof;
i) IL-7 or an active part or variant thereof, or a vector or a cell or a population of cells capable of producing IL-7 or an active part or variant thereof, or a nucleic acid molecule capable of encoding IL-7 or an active part or variant thereof; and ii) IL-33 or an active part or variant thereof, or a vector or a cell or a population of cells capable of producing IL-33 or an active part or variant thereof, or a nucleic acid molecule capable of encoding IL-33 or an active part or variant thereof;
i) IL-15 or an active part or variant thereof, or a vector or a cell or a population of cells capable of producing IL-15 or an active part or variant thereof, or a nucleic acid molecule capable of encoding IL-15 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof;
i) IL-7 and IL-15 or an active part or variant thereof, or a vector or a cell or a population of cells capable of producing IL-7 and IL-15 or an active part or variant thereof, or a nucleic acid molecule capable of encoding IL-7 and IL-15 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof;
i) IL-2 or an active part or variant thereof, or a vector or a cell or a population of cells capable of producing IL-2 or an active part or variant thereof, or a nucleic acid molecule capable of encoding IL-2 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof; and
i) IL-2 and IL-7 or an active part or variant thereof, or a vector or a cell or a population of cells capable of producing IL-2 and IL-7 or an active part or variant thereof, or a nucleic acid molecule capable of encoding IL-2 and IL-7 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof.

29. The pharmaceutical combination according to claim 27, wherein at least one of γc-cytokine or an active part or variant thereof in i), and/or at least one of IL-25 or IL-33 or an active part or variant thereof in ii) respectively, further comprise one or more modifications selected from the group consisting of fusion with a human immunoglobulin Fc fragment or a variant thereof; fusion with human serum albumin or a variant thereof; fusion with a tumor-penetrating peptide; fusion with a tumor-targeting single-chain or single-domain antibody; PEGylation; Xtenylation; PASylation; HESylation; or any other suitable chemical modifications.

30. The pharmaceutical combination according to claim 27, wherein the pharmaceutical combination is a recombinant protein comprising:
an amino acid sequence as set forth in any one of SEQ ID NOs: 1, 3, 4 and 5 or an active portion thereof; or an amino acid sequence having at least 70%, 80%, 90%, 95% or 99% identity with the amino acid sequence as set forth in any one of SEQ ID NOs: 1, 3, 4 and 5 or an active portion thereof; and
an amino acid sequence as set forth in SEQ ID NO: 7 or 8 or an active portion thereof, or an amino acid sequence having at least 70%, 80%, 90%, 95% or 99% identity with the amino acid sequence as set forth in SEQ ID NO: 7 or 8 or an active portion thereof.

31. The pharmaceutical combination according to claim 30, wherein the recombinant protein further comprises one or more peptide linkers that are cleavable in vivo or intratumorally; preferably, the peptide linkers are selected from the group consisting of one or more of the linker as set forth in any one of SEQ ID NOs: 21-29 and 32 or in vivo cleavable disulfide bond linker.

32. The pharmaceutical combination according to claim 27, wherein the pharmaceutical combination is a recombinant nucleic acid molecule comprising:
a first nucleotide sequence selected from the group consisting of: the nucleotide sequence as set forth in any one of SEQ ID NOs: 9, 11, 12 and 13, or a nucleotide sequence having at least 70%, 80%, 90%, 95% or 99% identity with the nucleotide sequence as set forth in any one of SEQ ID NOs: 9, 11, 12 and 13 or a variant thereof; and
a second nucleotide sequence selected from the group consisting of: the nucleotide sequence as set forth in SEQ ID NO: 15 or 16, or a nucleotide sequence having at least 70%, 80%, 90%, 95% or 99% identity with the nucleotide sequence as set forth in SEQ ID NO: 15 or 16 or a variant thereof.

33. The pharmaceutical combination according to claim 27, wherein the pharmaceutical combination is a vector comprising:
a first nucleotide sequence selected from the group consisting of: the nucleotide sequence as set forth in any one of SEQ ID NOs: 9, 11, 12 and 13, or a nucleotide sequence having at least 70%, 80%, 90%, 95% or 99% identity with the nucleotide sequence as set forth in any one of SEQ ID NOs: 9, 11, 12 and 13; and
a second nucleotide sequence selected from the group consisting of: the nucleotide sequence as set forth in SEQ ID NO: 15 or 16, or a nucleotide sequence having at least 70%, 80%, 90%, 95% or 99% identity with the nucleotide sequence as set forth in SEQ ID NO: 15 or

16.

34. The pharmaceutical combination according to claim 33, wherein the vector is selected from the group consisting of a plasmid or a viral vector, including but not limited to: an adenoviral vector, an adeno-associated viral vector, a retroviral vector, a lentiviral vector, a sleeping beauty or a Piggy Bac transposon system, CRISPRiCas9 knock-in system or any other suitable carriers.

35. The pharmaceutical combination according to claim 33, wherein the first nucleotide sequence and the second nucleotide sequence are present in separate vectors; or wherein the first nucleotide sequence and the second nucleotide sequence are present in the same vector.

36. The pharmaceutical combination according to claim 27, wherein the pharmaceutical combination is a cell or a population of cells comprising:
i) a cell or a population of cells capable of producing at least one of γc-cytokine selected from the group consisting of IL-2, IL-7, IL-9 and IL-15, or an active part or variant thereof; and
ii) a cell or a population of cells capable of producing at least one of IL-25 or IL-33, or an active part or variant thereof.

37. The pharmaceutical combination according to claim 36, wherein the cell is selected from the group consisting of an autologous cell or an allogeneic cell, including but not limited to: dendritic cells, T cells, macrophages, eosinophils, or any other suitable cells.

38. A method for preventing and/or treating a malignant tumor comprising administering to a subject in need thereof a prophylactically and/or therapeutically effective amount of a pharmaceutical combination comprising:
i) at least one of γc-cytokine or an active part or variant thereof, or a vector or a cell or a population of cells capable of producing at least one of γc-cytokine or an active part or variant thereof, or a nucleic acid molecule capable of encoding one of γc-cytokine or an active portion or a variant thereof; and
ii) at least one of IL-25 or IL-33 or an active part or variant thereof, or a vector or a cell or a population of cells capable of producing at least one of IL-25 or IL-33 or an active part or variant thereof, or a nucleic acid molecule capable of encoding at least one of IL-25 or IL-33 or an active part or variant thereof;
wherein at least one of γc-cytokine is selected from the group consisting of IL-2, IL-7, IL-9 and IL-15.

39. The method according to claim 38, wherein the pharmaceutical combination is selected from the group consisting of:
i) IL-7 and/or IL-9 or an active part or variant thereof, or a vector or a cell or a population of cells capable of producing IL-7 and/or IL-9 or an active part or variant thereof, or a nucleic acid molecule capable of encoding IL-7 and/or IL-9 or an active part or variant thereof; and IL-25 or an active part or variant thereof, or a vector or a cell or a population of cells capable of producing IL-25 or an active part or variant thereof, or a nucleic acid molecule capable of encoding IL-25 or an active part or variant thereof;
i) IL-7 or an active part or variant thereof, or a vector or a cell or a population of cells capable of producing IL-7 or an active part or variant thereof, or a nucleic acid molecule capable of encoding IL-7 or an active part or variant thereof; and ii) IL-33 or an active part or variant thereof, or a vector or a cell or a population of cells capable of producing IL-33 or an active part or variant thereof, or a nucleic acid molecule capable of encoding IL-33 or an active part or variant thereof;
i) IL-15 or an active part or variant thereof, or a vector or a cell or a population of cells capable of producing IL-15 or an active part or variant thereof, or a nucleic acid molecule capable of encoding IL-15 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof;
i) IL-7 and IL-15 or an active part or variant thereof, or a vector or a cell or a population of cells capable of producing IL-7 and IL-15 or an active part or variant thereof, or a nucleic acid molecule capable of encoding IL-7 and IL-15 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof;
i) IL-2 or an active part or variant thereof, or a vector or a cell or a population of cells capable of producing IL-2 or an active part or variant thereof, or a nucleic acid molecule capable of encoding IL-2 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof; and
i) IL-2 and IL-7 or an active part or variant thereof, or a vector or a cell or a population of cells capable of producing IL-2 and IL-7 or an active part or variant thereof, or a nucleic acid molecule capable of encoding IL-2 and IL-7 or an active part or variant thereof; and ii) IL-25 or an active part or variant thereof, or a vector or a cell or a population of cells capable of producing IL-25 or an active part or variant thereof, or a nucleic acid molecule capable of encoding IL-25 or an active part or variant thereof.

40. The method according to claim 38, wherein the malignant tumor is selected from the group consisting of fibrosarcoma, mucinous sarcoma, liposarcoma, chondrosarcoma, osteosarcoma, chordoma, angiosarcoma, endothelial sarcoma, lymphangiosarcoma, lymphangial endothelial sarcoma, synovial sarcoma, mesothelioma, Ewing's tumor, leiomyosarcoma, rhabdomyosarcoma, colon cancer, pancreatic cancer, breast cancer, ovarian cancer, prostate cancer, squamous cell carcinoma, basal cell carcinoma, adenocarcinoma, sweat gland cancer, sebaceous gland cancer, papillary carcinoma, papillary adenocarcinoma, cancer, bronchial carcinoma, medullary carcinoma, renal cell carcinoma, liver cancer, cholangiocarcinoma, choriocarcinoma, seminoma, embryonic carcinoma, nephroblastoma, cervical cancer, testicular tumor, lung cancer, small cell lung cancer, bladder cancer, epithelial cancer, glioma, astrocytoma, medulloblastoma, craniopharyngioma, ependymoma, pineal tumor, hemangioblastoma, acoustic neuroma, meningioma, oligodendroglioma, melanoma, neuroblastoma or retinoblastoma.

41. The method according to claim 38, wherein the pharmaceutical combination is a recombinant protein comprising:
an amino acid sequence as set forth in any one of SEQ ID NOs: 1, 3, 4 and 5 or an active portion thereof, or an amino acid sequence having at least 70%, 80%, 90%, 95% or 99% identity with the amino acid sequence as set forth in any one of SEQ ID NOS: 1, 3, 4 and 5 or an active portion thereof; and
an amino acid sequence as set forth in SEQ ID NO: 7 or 8 or an active portion thereof, or an amino acid sequence having at least 70%, 80%, 90%, 95% or 99% identity with the amino acid sequence as set forth in SEQ ID NO: 7 or 8 or an active portion thereof

42. The method according to claim 41, wherein the recombinant protein is administered in a dose of 0.0001 to 10 mg/kg body weight per time at the interval of 0 to 30 days.

43. The method according to claim 38, wherein the pharmaceutical combination is a vector comprising:
a first nucleotide sequence selected from the group consisting of: the nucleotide sequence as set forth in any one of SEQ ID NOs: 9, 11, 12 and 13, or a nucleotide sequence having at least 70%, 80%, 90%, 95% or 99% identity with the nucleotide sequence as set forth in any one of SEQ ID NOs: 9, 11, 12 and 13; and/or
a second nucleotide sequence selected from the group consisting of: the nucleotide sequence as set forth in SEQ ID NO: 15 or 16, or a nucleotide sequence having at least 70%, 80%, 90%, 95% or 99% identity with the nucleotide sequence as set forth in SEQ ID NO: 15 or 16.

44. The method according to claim 43, wherein the vector such as a virus is intratumorally administered in a dose of 10⁸-10¹³ pfu virus/tumor/time at the interval of 0 to 30 days.

45. The method according to claim 38, wherein the pharmaceutical combination is a cell or a population of cells.

46. The method according to claim 45, wherein the cell or the population of cells is administered in a dose of 10⁵-10¹¹ cells/kg body weight/time at the interval of 0 to100 days.
